(19) United States
(12) Patent Application Publication
(10) Pub. No.: US 2003/0170625 A1
(43) Pub. Date: Sep. 11, 2003

(54) Detection of differential gene expressions

(75) Inventors: Andre Rosenthal, Berlin (DE); Bernd Hinzmann, Berlin (DE); Reinhold Schaffer, Berlin (DE); Johannes Zuber, Berlin (DE); Oleg Tchernitsa, Berlin (DE); Martin Grips, Berlin (DE); Martin Hellriegel, Berlin (DE); Anne-Chantal Schmitz, Bonn (DE); Christine Sers, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: metaGen Gesellschaft fur Genomforschung mbH

(21) Appl. No.: 09/930,213

(22) Filed: Jan. 31, 2001

(30) Foreign Application Priority Data
Jan. 31, 2000 (DE) 100 04 102.7

(51) Int. Cl. C12Q 1/68; C07H 21/04

(52) U.S. Cl. 435/6; 536/23.2

(57) ABSTRACT

The invention relates to a process for detecting differential gene expressions and new nucleic acid sequences that can be obtained by this process.

### DESCRIPTION

The invention relates to a process for detecting differential gene expressions and new nucleic acid sequences that can be obtained by this process.

The genetic complexity of the cellular transformation on the level of the mRNA expression was first described more than 10 years ago (see, e.g., Groudine and Weintraub, PNAS USA 77 (1980), 5351-5354; Augenlicht et al., Canc. Res. 47 (1987), 6017-6021). Global sequence data with respect to the gene activity altered in the pathogenesis of human cancer were obtained only recently, after new methods for determining gene expression profiles were developed (Zhang et al., Science 276 (1997), 1268-1271; Chang et al. Oncogene [oncogenes] 16 (1998), 1921-1930; by Stein et al., Nucleic Acids Res. 25 (1997), 2598-2602). Complex gene expression profiles on which the numerous tumor-specific cell functions are based are adjusted at least partially by the accumulation of multiple genetic alterations in the abnormally activated signal transduction methods and transcription factors (Fearon and Vogelstein, Cell 61 (1990), 759-767; Stanbridge, Annu. Rev. Genet. 24 (1991), 615-657).

A more important aspect of the multistage tumorigenesis is the activation of members of the Ras-gene family that is produced by mutations. Ras-mutations are associated with an adverse tumor prognosis (Mao et al., Cancer Res. 54 (1994), 1634-1637; Sasaki et al., Cancer Res. 53 (1993), 1906-1910; Yaginuma et al., Gynecol. Oncol. 46 (1992), 45-50; Ahnen et al., Cancer Res. 58 (1998), 1149-1158). Ras-mutations are especially common in the case of sporadic tumor diseases of the pancreas, colon, lungs and the myeloid system (Boss, Canc. Res. 49 (1989), 4682-4689).

The Ras-gene products are small GTP-binding proteins that influence the transcription globally by acting as “main switches” in signal transduction processes, in which extracellular signals are linked with processes in the nucleus (Abdellatif et al., J. Biol. Chem. 269 (1994), 15423-15426; Malumbres and Pellicer, Frontiers in Bioscience 3 (1998), 887-912). In normal cells, the concentration of Ras-GTP in reaction to the binding of growth factors, cytokines or other ligands of membrane-bonded tyrosine kinase receptors increases transiently. The conversion into the inactive, GDP-bonded form of Ras is carried out by its low intrinsic GTP-hydrolysis activity and is accelerated by additional Ras-GTPase-activator proteins (GAP). Oncogenes, in the amino acid codons 12, 13, 59 or 61-mutated forms of Ras, are insensitive to GAP-stimulation and are consequently held in their active state (for an overview, see Malumbres and Pellicer (1998), supra, Marshall, FASEB J. 9 (1995), 1311-1318; MacDonald and McCormick in: Oncogenes and Tumour Suppressors, Eds. Peters, G. and Vousden, K. H., 121-153, Oxford University Press, Oxford 1997). The Ras-GTP amounts are increased even in tumors that contain no activator mutations (Patton et al., Cancer Res. 58 (1998), 2253-2259; Clark and Der, Breast Cancer Res. Treat. 35 (1995), 133-144). In the absence of intrinsic mutations, the Ras-signal transduction method can be stimulated by inactivating mutations of the Ras-regulator NF-1 GAP in the neurofibromatose type I (DeClue et al., Cell 69 (1992), 265-273) by complexing of effector proteins that have an upstream effect with the Bcr-Abl protein tyrosine kinase in chronic myelogenic leukemia (Puil et, al., EMBO J. 13 (1994), 764-773) and by direct association of Ras with the STP-C488 protein of the DNA tumor virus herpes saimiri (Jung and Desrosiers, Mol. Cell. Biol. 15 (1995), 6506-6512).

On the cellular level, two main changes are observed as a result of the Ras-activation: mitogenesis and reorganization of the cytoskeleton. A permanent activation of Ras produces an enhancement of the normal cellular reaction. Essential signals for the cellular transformation, invasiveness, angiogenesis and metastasizing are transduced downstream from Ras by branched signal paths. These signal paths comprise the Raf/Mek/Erk cascade of cytoplasmatic kinases, the signal path that contains the small GTP-binding proteins Rac/Rho, the PI3 kinase signal path, i.a. Via these signal paths, various transcription factors, such as, for example, Elk 1, SRF, Jun, ATF2 and NFkB, are stimulated (for an overview, see, e.g., Khosravi et al., Adv. Cancer Res. 72 (1998), 57-107).

In view of the complexity of the non-linear Ras-signalling and the extremely large number of potential targets, there is a great need to provide a process that allows a determination of the transcriptional changes that are associated with a Ras-transformation. In addition, this process should also be used for the analysis of transcriptional changes that are produced by other processes.

To solve this problem, the concentrations or amounts of individual transcripts in phenotypical normal 208 F rat fibroblasts (Quade, Virology 98 (1979), 461-465) were determined with those in the H-Ras-transformed cell line FE-8 derived from, 208 F-cells (Greigel et al., Int. J. Canc. 38 (1986), 697-705) using a PCR-based cDNA substraction method, subtractive suppression hybridization (Diatchenko et al., PNAS USA 93 (1996), 6025-6030). This process allowed, surprisingly enough, an efficient isolation of known genes—and of special importance—the isolation of new sequences. The two cell lines that are used are closely related, quasidiploid cell lines to keep transcriptional changes as few as possible based on structural and numeric chromosomal aberrations, which are not directly associated with the Ras-induced transformation. The cDNA fragments (n=1257) that are obtained in this way after forward- and backward-substraction were sequenced and arranged in an array. After reverse or conventional Northern Analysis, an H-Ras-specific expression profile that comprises new sequences (n=45), expressed sequence tags (n=104) and known genes (n=244) was established. Then, this gene profile was used for the comparison of mRNA expressions between H-Ras-transformed 208 F-cells and with cells that were transformed by two other tumor-associated Ras-isoforms K-Ras and N-Ras. Moreover, target genes (n=61) were identified, whose transcriptional changes are adjusted by the Ras/Raf/Mek-signal paths.

A first aspect of the invention relates to a process for determining transcriptional changes in a cell, especially in a mammal cell, such as, for example, a rodent cell or a human cell, associated with a physiological change, e.g., a transformation, preferably a Ras-mediated transformation, relative to another cell that does not exhibit this specific physiological change. The process for differential transcription analysis is characterized in that mRNA is obtained from a first cell and a second cell, a subtractive suppression hybridization is performed with the mRNA that is obtained, and a population of genes expressed differentially in both cells is identified, whereby the first and the second cell are distinguished relative to a physiological change that is to be examined. In addition, the process preferably comprises a verification of the differential expression, preferably by reverse and/or conventional Northern Blot analysis.

As a first cell, preferably a transformed cell is used, and as a second cell, preferably a non-transformed cell is used. An Ras-transformed cell is especially preferably used as a first cell, and a non-transformed cell is especially preferably used as a second, whereby as a Ras-transformed cell, especially a cell that is transformed with a mutated Ras-gene product, for example an H-Ras-, K-Ras- or N-Ras-transformed cell, is used.

The first cell and the second cell preferably are derived from the same species, especially a mammal, such as, for example, a rat, mouse, human, etc. In addition, the first cell and the second cell are preferably derived from the same cell type, for example fibroblasts. Moreover, it has proven advantageous to use a first cell and a second cell that essentially have no chromosomal aberrations that could possibly distort the pattern of the differential gene expression caused by the physiological change that is to be examined, e.g., the Ras-transformation.

Subtractive suppression hybridization is a technique based, on nucleic acid amplification, e.g., a PCR-based technique, which comprises a reverse transcription step, in which the differentially expressed transcripts are converted into cDNA molecules. Another characterization of the differentially expressed genes can comprise an at least partial sequence analysis of the identified cDNA molecule and an adjustment with sequence data bases such as Genbank, EMBL or EST.

By the process up until now, 1257 cDNA sequences were obtained by forward-subtraction (normal 208 F-cells minus transformed FE-8 cells) and backward-subtraction (transformed FE cells minus normal 208 F cells). From this, a total of 823 individual sequences were identified, of which 427 correspond to known genes and 303 correspond to expressed sequence tags. To date, 93 sequences are unknown. The differential expression of 39.3 (47.8%) genes and gene fragments was verified by Northern Analysis. Moreover, 236 cDNA sequences corresponding to only very small concentrations of transcripts that occur were obtained. With high probability, a portion of these sequences (>100) also has a differential expression.

The population of preferably at least 100 differentially expressed genes that can be obtained by the process according to the invention using cDNA subtraction can be used in a new way, e.g., by arrangement in arrays, to study the relationship between a signalling molecule and a transcription target on the level of transcription. In this way, the number of biologically relevant targets in the studied cells can be reduced to a limited number of genes that then can be thoroughly examined on their involvement in specific aspects of the respective physiological change, e.g., the tumorigenesis.

A subject of the invention is a nucleic acid, characterized in that it shows a differential expression in tumor cells and normal cells, comprising

(a) one of the nucleic acid sequences shown in FIG. 11,

(b) partial sequences thereof with a length of at least 50, preferably at least 100, and especially preferably at least 200 nucleotides,

(c) a sequence that hybridizes with a sequence that consists of (a) and/or (b) under stringent conditions, and/or

(d) a sequence that is complementary to a sequence that consists of (a), (b) and/or (c).

The process according to the invention makes it possible to identify genes whose expression in the Ras-transformed cells is enhanced in comparison to the non-transformed cells. These genes preferably comprise the corresponding nucleic acid sequences (T-clones) that are indicated in FIG. 11 or partial sequences thereof with a length of at least 50, preferably at least 100 and especially preferably at least 200 nucleotides.

In addition, the process according to the invention makes it possible to identify genes whose expression in the Ras-transformed cells is reduced in comparison to the non-transformed cells. These genes preferably comprise the corresponding nucleic acid sequences (N-clones) that are indicated in FIG. 11 or partial sequences thereof with a length of at least 50, preferably 100 and especially preferably at least 200 nucleotides.

In addition to the nucleic acid sequences that are indicated in FIG. 11 and partial sequences thereof, the invention also comprises nucleic acid sequences, which hybridize under stringent conditions with one of the nucleic acid sequences indicated in FIG. 11 or partial sequences thereof (as indicated above). The term “hybridization” according to this invention is used as in Sambrook et al., Molecular Cloning. A Laboratory Manual, Cold Spring Harbor Laboratory Press (1989), 1.101-1.104). According to this invention, we thus speak of hybridization under stringent conditions, if, after washing for one hour with 1×SSC and 0.1% SDS at 55° C., preferably at 62° C. and especially preferably at 68° C., especially for 1 hour in 0.2×SSC and 0.1% SDS at 55° C., preferably at 62° C. and especially preferably at 68° C., a positive hybridization signal is observed. This invention comprises a nucleotide sequence that hybridizes under such washing conditions with a nucleotide sequence shown under FIG. 11.

In particular, this invention also includes genes that are homologous to the nucleic acid sequences shown in FIG. 11, especially homologous genes from other species, especially human genes, or allelic variations of these genes. Such sequences preferably hybridize under the above-indicated conditions with the nucleic acid sequences that are shown in FIG. 11.

The genes that are identified by the process according to the invention or their transcripts or gene products are suitable as targets for diagnostic or therapeutic purposes, especially for tumor diagnosis or tumor therapy. Diagnostic applications comprise a qualitative or quantitative determination of the presence, the amount, the activity or the localization of the nucleic acid or the gene product according to known methods. Therapeutic applications comprise, for example, a modulation of the expression of the nucleic acid, e.g., by gene-therapy administration of nucleic acid or an administration of antisense-RNA or ribozymes. In addition, the amount, activity and/or localization of the polypeptide coded by the nucleic acid can also be modulated, e.g., by administration of the polypeptide or an activator thereof or by administration of an antibody that is directed against the polypeptide, e.g., in the form of a conjugate with radioisotopes or cytotoxic substances, or an inhibitor of the polypeptide.

In addition, the process according to the invention makes it possible to identify genes that show a differential expression in cells transformed by different Ras-isoforms. The gene expression was thus examined in different cells that respectively express one of the three predominant mutated Ras-isoforms H-Ras, K-Ras and N-Ras. In this case, an identical pattern of positive and negative changes for 237 genes (90%) was found. 26 genes, however, showed an isoform-specific expression pattern.

In addition, the process according to the invention makes it possible to identify genes that show a differential expression in cells that are treated with an active substance and untreated cells, especially tumor cells. As active substances, basically any substances, especially pharmacologically active substances that show an effect on the transcription in the cell, can be used. Preferred examples of active ingredients are modulators, i.e., activators or inhibitors of tumorigenesis. Especially preferably modulators of the Ras-activity are used.

In this embodiment of the process according to the invention, the influence of active substances that have been added to the first and/or second cell can be tested on the population of differentially expressed genes. When MAP-kinase inhibitor PD98059 was added to a Ras-transformed cell, it was found, for example, that the transcription of 61 genes is significantly decreased in comparison with untreated cells and was attributed at least partially to the level before the transformation.

The sensitivity of the transcriptional modulation of these genes with respect to the inhibition of a signalling by the MAP-kinase defines a subclass of Ras-sensitive targets, which are adjusted by substrates of Erkl/Erk2 and are probably directly responsible for the transforming properties of the cell. The 116 genes that are not affected by the inhibitor are presumably adjusted by MEK-independent signal transduction paths downstream from the Ras.

Surprisingly enough, it was possible to identify an unexpectedly high number of genes, which can counteract a malignant proliferation, by the process according to the invention. These genes that are identified by the differential, expression analysis are tumor suppressor genes of Class II, since they are not a primary target of tumor-initiating mutations. Instead of this, the genes of Class II are distinguished in that their expression is adjusted by genes of Class I that code for transcriptional regulators, such as, for example, oncogenic transcription factors or repressors, and can be the subject of a mutation. An enhancement of expression of transformation suppressor genes of Class II can block the transformed phenotype of Ras-expressing cells (Sers et al., J. Cell. Biol. 136 (1997), 935-944). A functional connection between a permanently activated Ras-signalling and the repression of Class II suppressor activity thus exists.

Notably, it was possible to find more than ten negative growth regulators in an independent expression profile by subtraction of 208 F cDNA (driver) from REF-52 cDNA (tester). REF-52 cells are distinguished in that they activate a premature aging program in reaction to H-Ras expression (Serrano et al., Cell 88 (1997), 593-602) and have high mRNA concentrations of negative regulators, which are not expressed in 208 F-cells.

In addition, the invention is explained by the figures and examples below.

### DESCRIPTION OF THE FIGURES

FIG. 1 shows an overview of the DNA libraries obtained after forward- and backward-subtractions. Sequence analysis:

homology studies were performed with use of the BLASTN program against the NCBl Non-Redundance and EST data bases. Similarities relative to a data base sequence were defined as a sequence identity of >95% over a region of 150 to 1000 bp as a function of the cDNA insert length.

FIG. 2 shows an overview of differentially expressed sequences confirmed by reverse (R) and/or conventional Northern Blot analysis (N, T). Sequence identity, species and access number are listed in order of best correspondence in the Blast analysis. Species abbreviations: H, human; M, mouse; R, rat; C, chicken; HA, hamster; X, Xenopus laevis. Redundancy means the number of individual cDNA clones, which correspond to an identical gene in the BLAST analysis. Numbers N1-N70 correspond to the genes that are adjusted down by Ras-transformation as shown by Northern Blot in FIG. 7. Numbers T1-T74 correspond to genes that are adjusted up as shown in FIG. 7. The amounts of mRNA were analyzed by densitometry. The indicated numbers correspond to the ratio of densitometric values (volume analysis) of 208F compared to FE-8 mRNA (extent of downward-adjustment, left section) and FE-8 compared to 208F mRNA (extent of upward-adjustment, right section). A value of 30 or more shows that a transcript cannot be detected in one of the studied cells. The following cDNA fragments were not detectable in the case of reverse or conventional Northern Blot analysis. 208 F specific clones: p190-B (access no. U170032, SLIT-2 (AF141386), Slugh Zinkfinger (U79550), Semaphorin E (AB000220), GLE-1 (AF058922), TID1 (AF061749), ARF-GEP1 (AF023451), DEAD Box RNA helicase-like protein (NM-004398); FE-8-specific clone; G21 protein (AF131207), p68 RNA helicase (X 65627), LZTR-1 (D38496), Smcx (Z29651), SHMT (L11932) and CRK SH3-protein/C3G (D21239)

FIG. 3 shows Ras-target genes, which react to an MEK-inhibition by PD98059. Left column: preferentially expressed in normal 208 F-cells and genes that are adjusted down in H-Ras-transformation; right column: genes up-adjusted in H-Ras-transformation. Transcript amounts: O, mRNA not detectable in Northern Blots with whole RNA; +to +++, low, medium or high mRNA expression. The sequences that are referred to as a to e were used as samples in the Northern Blots shown in FIG. 8.

FIG. 4 shows specific changes in transcriptional Ras-isoforms. Transcript amounts: O, mRNA not detectable in Northern Blots with whole RNA, +to ++++, low, medium, high or very high RNA expression. The sequences that are referred to as f to i were used as samples in the Northern Blots shown in FIG. 8.

FIG. 5 shows properties of cells that were used for the identification of Ras-transformation targets.

a) Morphology of normal 208 F fibroblasts and H-Ras-transformed FE-8 cells untreated and incubated with the MEK-inhibitor (PD98059). Phase contrast, 100×magnification.

b) DNA histogram of 208F and FE-8 cells obtained with continuous-flow cytometry. Abscissa: fluorescence intensity; ordinate: numbered cells; the numbers relate to the proportion of cells (%) in various phases of the cell cycle. The 208 F and FE-8 rat cells showed a pseudo-diploid karyotype without significant numerical chromosomal aberrations.

c) Anchor-independent proliferation of cells in cultures on poly-hema-coated surfaces. Ordinate: absorption at 490 nm.

d) Western Blot analysis of the 21-Ras expression and

e) The Phospho-p44/42 MAPK expression.

FIG. 6 shows a Reverse Northern Blot analysis of subtracted cDNA fragments. Representative example of 93 arranged ESTs, which were obtained from a 208F-FE-8 subtracted library (N-clone).

a) Hybridization sample: ³²P-labeled 208 F cDNA,

b) Hybridization sample: ³²P-labeled FE-8 cDNA. Control-DNAs: cloning vector PCR2.1 (filter position D22), GAPDH (D23), H-Ras (D24).

FIG. 7 shows a conventional Northern Blot analysis of preferentially expressed genes. Upper half, clones N1-N70 corresponding to genes adjusted down in H-Ras-transformed FE-8 cells were used as hybridization samples. Lower half, clones T1-T74 corresponding to genes up-adjusted in FE-8 cells were used as hybridization samples. Left column, individual blots of whole RNA from normal 208 F cells, right column, from FE-8 RNA. Black arrows show the correct transcript size as described in the literature. White arrows show aberrant transcripts. Control hybridizations were carried out with a GAPDH sample (A to E). Original size of the Northern Filter; 3×1 cm.

FIG. 8 shows representative examples of the actions of the Ras/Raf/Mek signal transduction path and various Ras isoforms in the target gene transcription.

a to e) Northern Blot analysis of the mRNA expression in normal 208 F fibroblasts, H-Ras-transformed A cells and FE-8 cells treated with PD98059.

f to i) Northern Blot analysis of the mRNA expression in normal 208 F cells and in 208F cells transformed with mutated H-Ras, K-Ras and, N-Ras.

k) MMP-3, representative example of a gene without significant differential expression.

FIG. 9 shows a characterization of cells transformed with isoforms of the Ras oncogene.

a) Morphology of normal 208F fibroblasts, H-Ras-transformed FE-8 cells, K-Ras- and N-Ras-transformed 208F cells. Phase contrast, 100×magnification.

b) Anchor-independent proliferation of cells in cultures on poly-hema-coated surfaces. Ordinate: absorption at 490 nm.

c) Western Blot analysis of Ras expression.

FIG. 10 shows representative examples of a differential mRNA expression in stable rat ovarian surface epithelial cells transformed with K-Ras and in conditional H-Ras-transformed fibroblasts. Left: Northern Blot analysis of whole RNA from normal rat ovarian surface epithelial cells (ROSE199) and 2 K-Ras-transformed derivatives (ROSE A2/1 K-Ras and ROSE A2/5 K-Ras). Right: RNA from 208F cells, stable transformed FE-8 cells and conditionally transformed 208F-iHRas-cells before (−IPTG) and after 4 days of a Ras-induction (+IPTG).

FIG. 11 shows the nucleotide sequences of cDNAs corresponding to the identified differentially expressed transcripts. In addition to the concretely indicated sequences, FIG. 11 also contains, of course, a disclosure relative to the complementary sequences.

FIG. 12 shows the nucleotide sequences of homologous human cDNAs. FIG. 12 also contains a disclosure relative to the complementary sequences.

FIG. 13 shows a correlation of nucleotide sequences (rats) shown in FIG. 11 to the homologous human sequences according to FIG. 12.

SEQ. ID NOS. 1-885; sequences SEQ. ID NOS. 1, 2, 3 . . . 335 correspond to sequences 1, 2, 3 . . . 335 according to FIG. 12. Sequences SEQ. ID NOS. 336, 337, 338 . . . 632 correspond to sequences nos. 1, 2, 3 . . . 297 according to FIG. 11, and sequences SEQ. ID NOS. 633, 634, 635 . . . 885 correspond to sequences T1, T2, T3 . . . T253 according to FIG. 11.

### EXAMPLES

1. Methods

1.1 Cell Culture and DNA Transfections

Cells were cultivated in Dullbeco's Modified Eagle's Medium (DMEM) supplemented with 10% fetal calf serum. The transfections were carried out by calcium phosphate precipitation as described in Griegel et al. (Int. J. Canc. 38 (1986), 697-705). To establish N-Ras transformants, 208F cells were co-transfixed with pcDneo and the N-Ras (G12D) oncogene (Souyri et al., Virology 158 (1987), 69-78) and selected in DMEM with 400 μg/ml of G418. The K-Ras (C12V) oncogene was cloned from human colon cancer cell line SW480 and transfixed in 208F cells. 208F-K-Ras cells were isolated from morphologically transformed transfectants. FE-8 cells are G418-resistant H-rus (G12V)-transformed derivatives of 208F (Griegler et al., supra).

For production of subtracted libraries, cells from an early isolate of the FE-8 cell line were used. 208F cells were kept in culture no longer than 30 days after transfection. K-Ras-transformed rat ovarian surface epithelial cells were isolated with K-Ras (C12 V) after transfection of ROSE199 cells (Adams and Auersperg, Exp. Cell Biol. 53 (1985), 181-188). For the production of 208F-iH-Ras cells, which show an inducible expression of the H-Ras oncogene, 208F cells were co-transfixed with the plasmids pSVlacOras and pHβlacINLSneo (Liu et al., Canc. Res. 52 (1992), 983-989) and selected in standard medium with 400 μ/ml of G418. For the Ras expression, the cells were incubated for four days with 20 mmol of isopropyl-1-thio-β-D-galactoside (IPTG).

The MEK inhibitor PD98059 (Dudley et al. PNAS USA 92 (1995), 7686-7689) was dissolved in DMSO at a final concentration of 50 mmol. FE-8 cells were treated for 2 days with PD98059 at a final concentration of 50 μmol. The anchor-independent proliferation was determined in a semiquantitive manner in cultures that were grown on microtiter plates coated with poly-2-hydroxyethylmethacrylate (poly-hema; Sigma). 75 μl of a poly-hema-stock solution (5 mg/ml in 95% ethanol) was added into the recesses and allowed to dry for 72 hours at 37° C. Cell suspensions were saturated in coated plates (1000 cells/recess), and the growth was determined after 5 days with use of an XTT assay (Roche, Mannheim, Germany).

1.2 Cloning of Differentially Expressed Sequences by Subtractive Suppression Hybridization (SSH)

Whole RNA was obtained from subconfluent cultures as described by Chomczynski and Sacchi (Anal. Biochem. 162 (1987), 156-159). mRNA was isolated from 1 mg of whole RNA with use of the mRNA separator kit (Clontech, Palo Alto, Calif., USA). cDNA synthesis and subtraction were performed with use of the PCR-Select™ subtraction kit (Clontech, Palo Alto, Calif., USA) according to the manufacturer's instructions with the following modifications: a driver/tester volume ratio of 2:12 was used in the first hybridization. 26 cycles of the primary PCR and 10 cycles of the secondary PCR were performed with use of the Advantage CDNA Polymerase Mix (Clontech). To determine the efficiency of the cDNA subtraction, transcript amounts of the constitutively expressed gene GPADH were compared by RT-PCR in subtracted and unsubtracted populations of 208F RNA or FE-8 RNA. The detection of the GAPDH sequences for both subtractions required 28 PCR cycles with use of subtracted cDNA as matrices, while only 18 cycles were required for amplification of GAPDH from control cDNA. In addition, the amounts of genes for which a differential expression in 208F and FE-8 cells is known were tested by RT-PCR. As expected, H-Ras-specific cDNA was concentrated in subtracted and unsubtracted FE-8 cDNA. The amount of lysyloxidase cDNA was higher in subtracted 208F cDNA than in unsubtracted 208F cDNA and showed a reduction by a small amount in unsubtracted FE-8 cDNA up to a no longer detectable amount in subtracted FE-8 cDNA.

The subtracted cDNA sequences were purified with use of the QIA Quick PCR purification kit (Quiagen, Valencia, Calif., USA). 10 ng of cDNA were inserted in the vector pCR2.1 (invitrogen, Leeg, The Netherlands) by T/A cloning. Individual transformants with cDNA fragments were isolated from white colonies on X-GaI/IPTG-agar plates. To determine the quality of the library with respect to redundancy and specificity, 35 randomly picked cDNA transformants from each DNA library were isolated and sequenced. The differential expression of the inserted sequences was analyzed in the Nothern Blots with 10 μg of whole RNA from 208F and FE-8 cells.

1.3 Sequence Analysis

Sequencing reactions were performed with the M13 Universal Primer with use of the BigDye sequencing kit (Perkin Elmer) according to the manufacturer's instructions. The sequences were determined in an AB1377 sequencing device. The sequencing of, the cDNA insertions of subtracted libraries was completed when the number of redundant sequences significantly exceeded those of new clones. The clustering was carried out with use of GAP4 software (Staden Package). Sequence homology studies were carried out from the data bases GenBank (nr) and Expressed Sequence Tag (dbest) with use of the BLASTN program in NCBI (http:\\www.ncbi.nlm.nih.gov\BLAST).

1.4 Hybridization Analysis

Non-redundant plasmid-DNA samples of all identified fragments were transferred onto 96-hole-microtiter plates. With use of PCR-Select™ adaptor-specific primers, a PCR amplification was performed with 30 cycles (30 sec 94° C., 30 sec 68° C., 90 sec 72° C.). The average size of the inserted fragments was 800 bp. The PCR-amplified insertions were plotted on 2 25×12 cm Nytran nylon membranes respectively (Schleicher and Schuell, Dassel, Germany). A Reverse Northern Analysis was performed as described by Stein et al. (Nucleic Acids Res. 25 (1997), 2598-2602), except that the following hybridization conditions were used: Pre-hybridization of the membranes was carried out with 5×Denhardt's reagent, 5×SSC, 50 mmol of phosphate buffer, 0.5% SDS, 100 ng/ml of tRNA at 65° C. for 3 hours. The hybridization was carried out in the same buffer without Denhardt's reagent and 50 mmol of phosphate buffer at 65° C. for 16 hours.

For the conventional Northern Blot analysis, 10 μg of whole RNA was electrophoretically separated in 1% agarose gel with formaldehyde and blotted in 20×SSC on Protran nitrocellulose membranes (Schleicher and Schuell, Dassel, Germany). The cDNA fragments were labeled with ³²P-dCTP with use of the Ready Prime System (Amersham, Brunswick). The hybridization was carried out in ExpressHyb hybridization buffer (Clontech) at 68° C. overnight. The membranes were washed twice in 2×SSC, 0.1% SDS at 42° C. for 20 minutes and 2×0.1 SSC, and 0.1% SDS at 66° C. for 30 minutes and analyzed by autoradiography.

2. Results 2.1 Properties of the Cells Used for Identifying Ras-transformation Targets

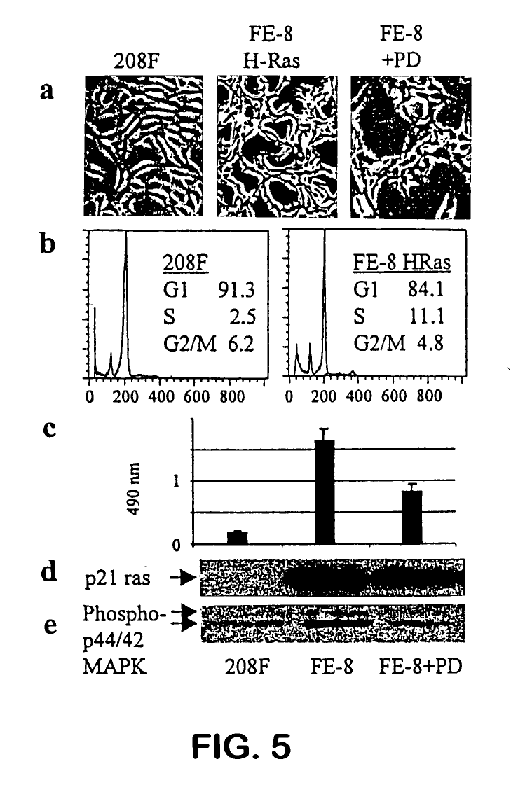

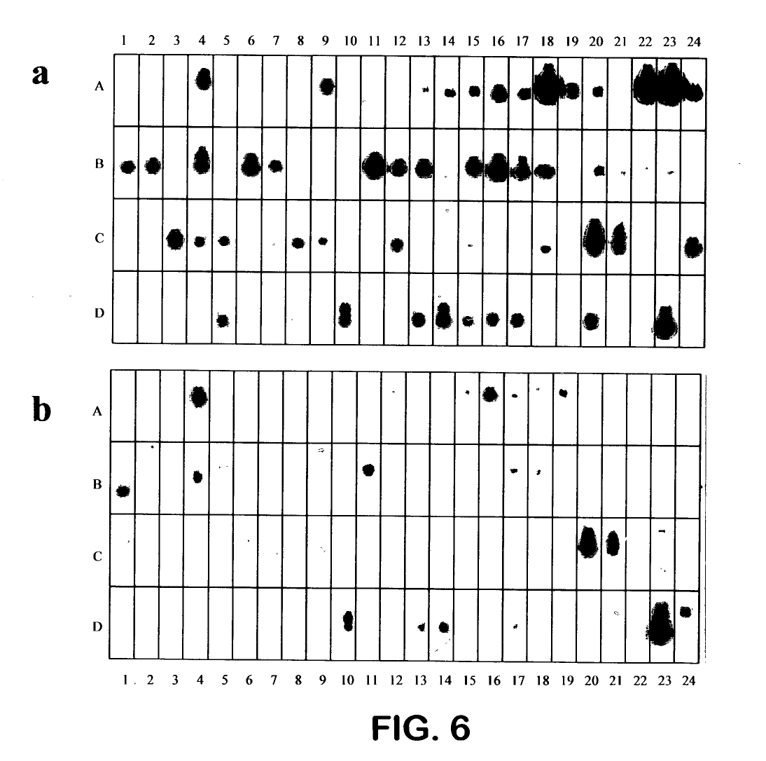

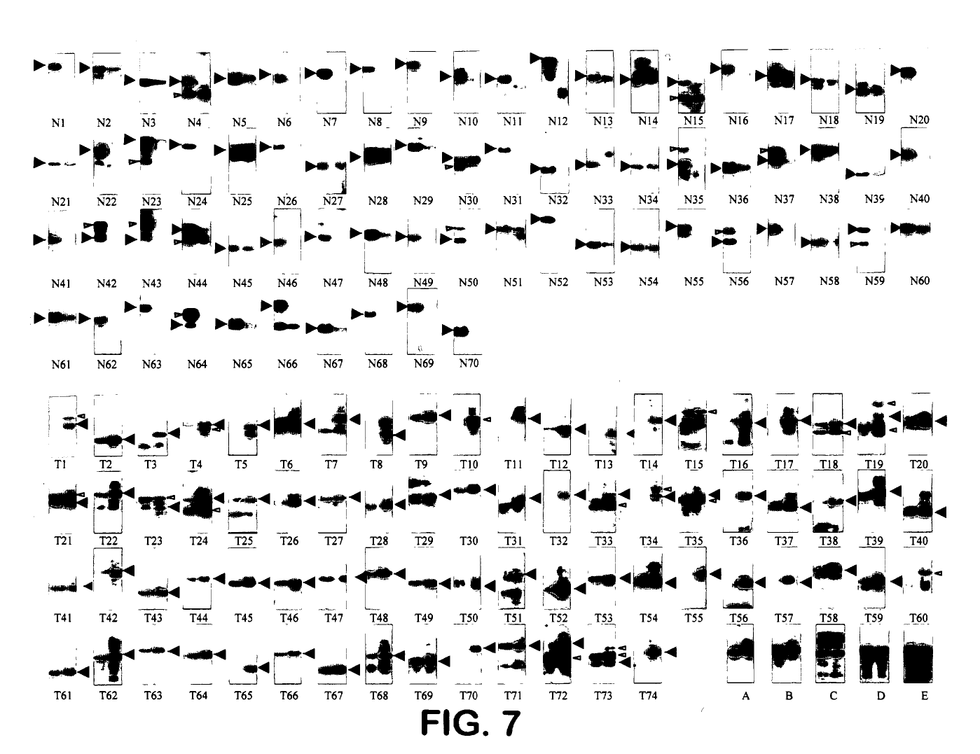

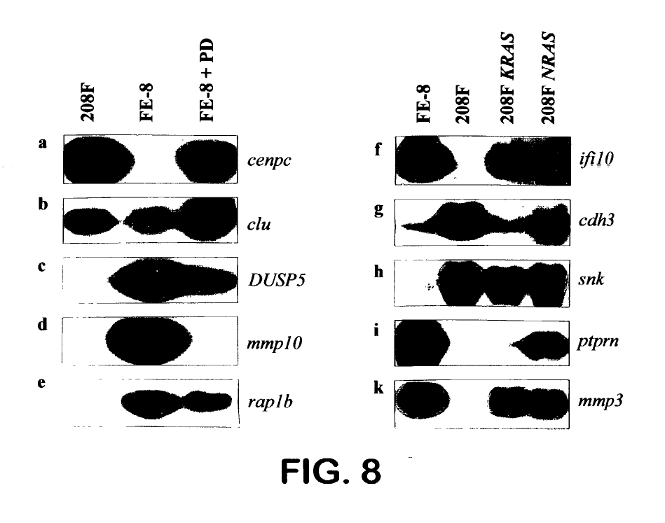

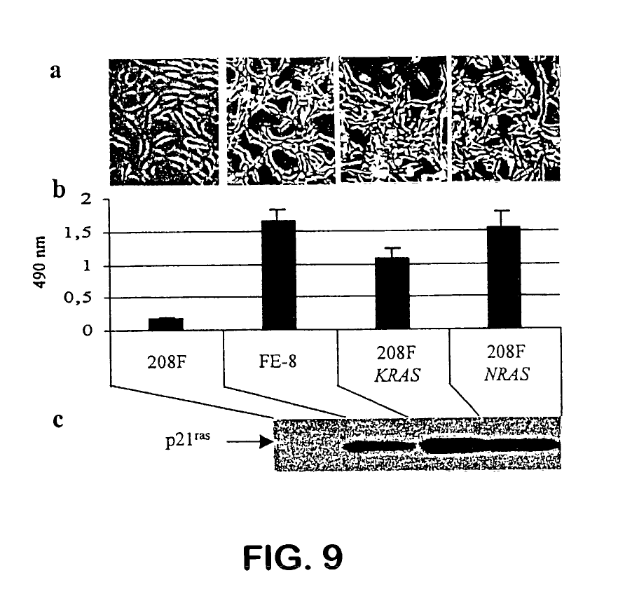

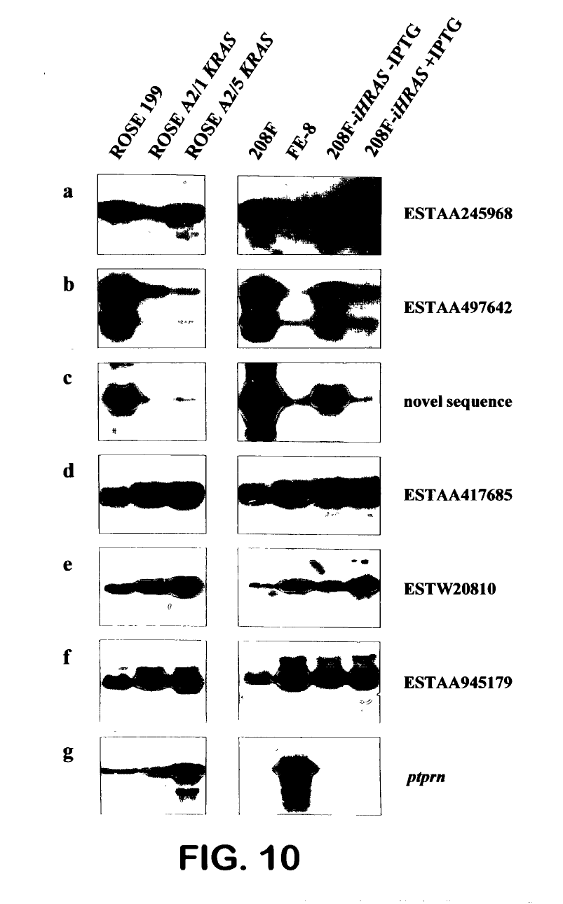

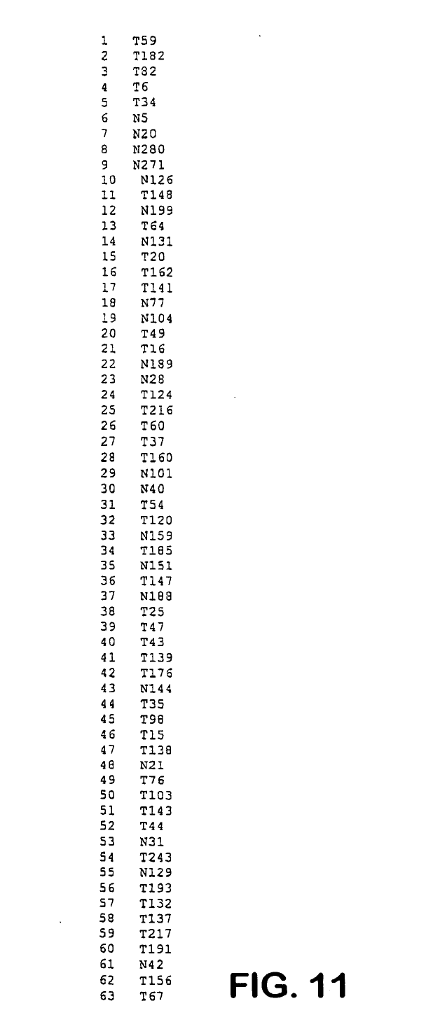

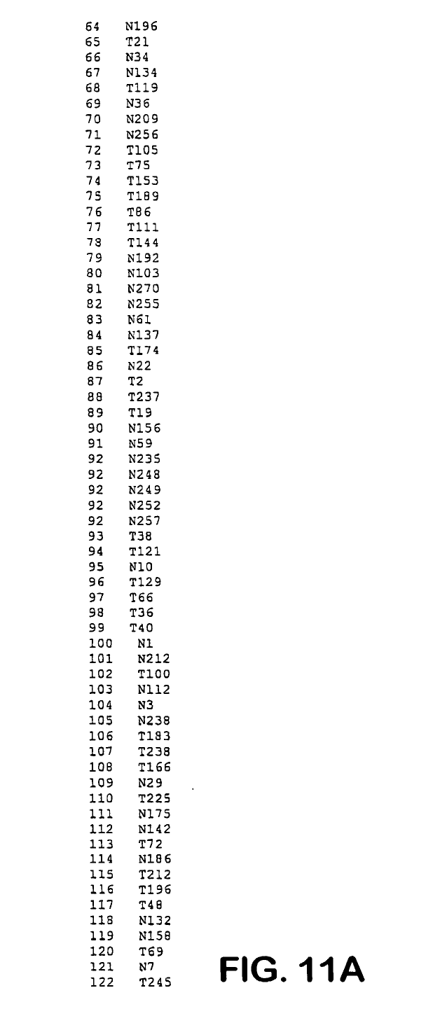

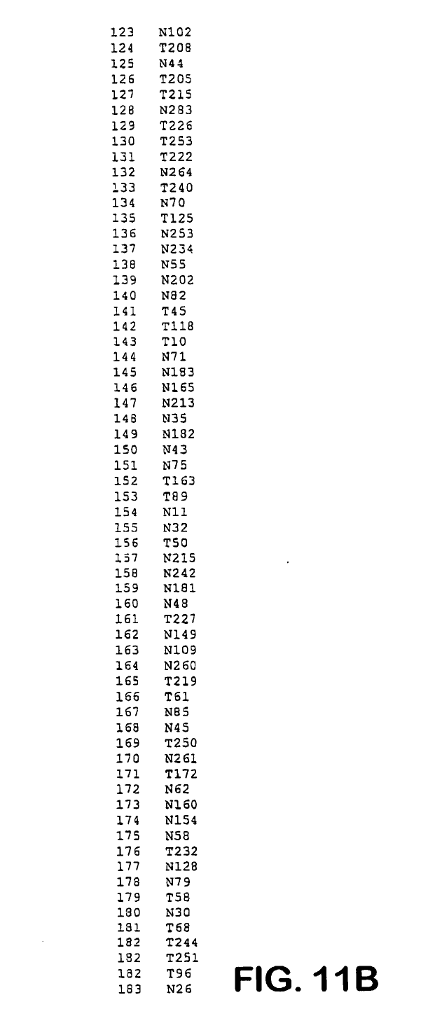

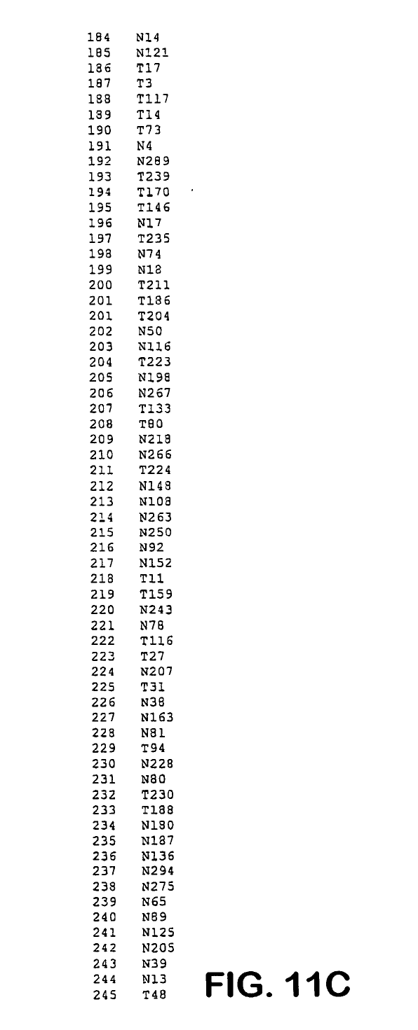

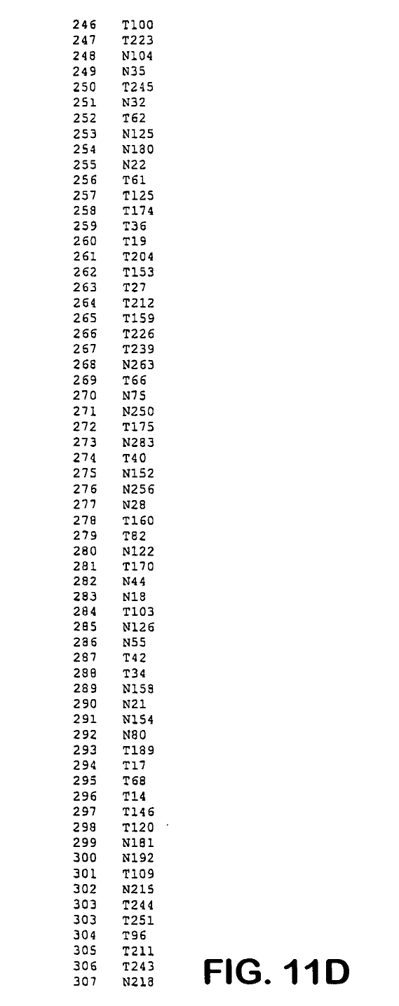

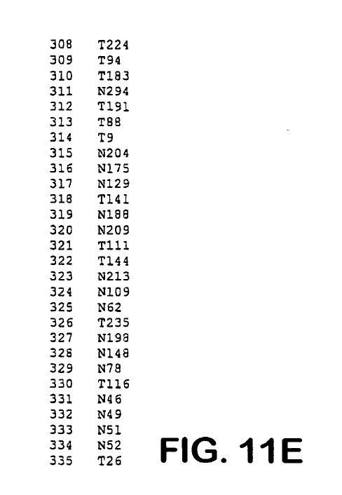

The preneoplastic cell line 208F and its malignant Ras-transformed derivative FE-8 (FIG. 5a) show an almost diploid karyotype without significant numeric or structural chromosomal aberrations (FIG. 5b). While 208F cells do not show any spontaneous transformation, FE-8 cells are anchor-independent (FIG. 5c) and show a strongly malignant action with subcutaneous injection in athymic hairless mice or newborn rats (Griegel et al. (1986), supra; Sers et al. (J. Cell Biol. 136 (1997), 935-944). The 208F/FE-8 cell system thus is suitable for the preparation of a population of transcriptionally altered genes, which are caused by a permanent Ras-signalling and by changes in the general gene adjustment.

2.2 Isolation of Sequences Expressed Differentially in Transition From the Normal State to the Transformed State

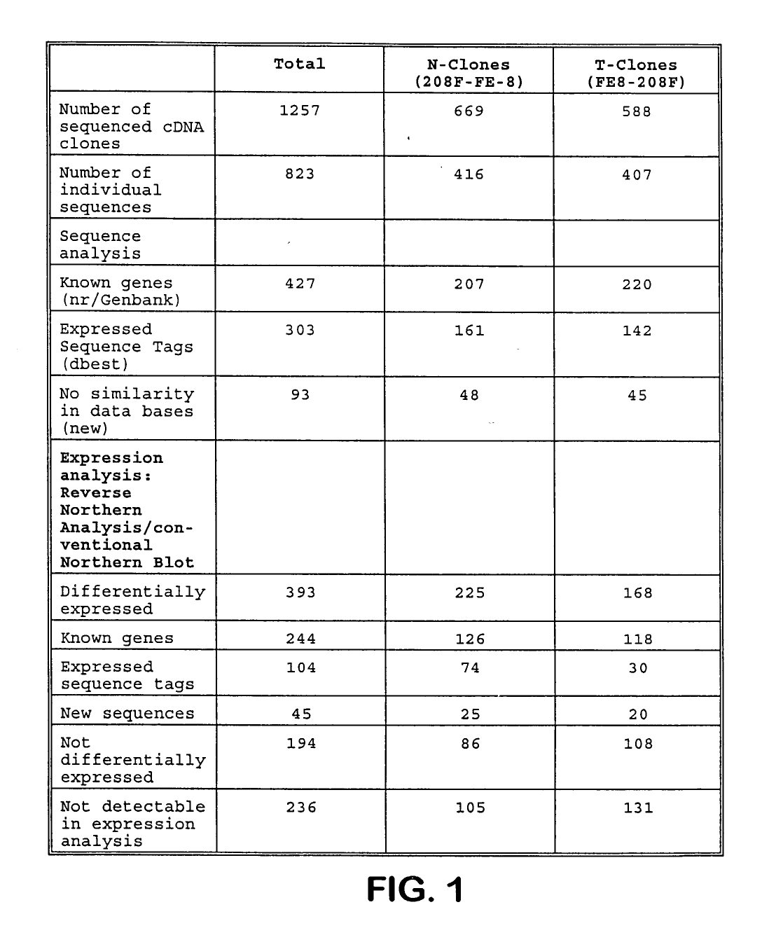

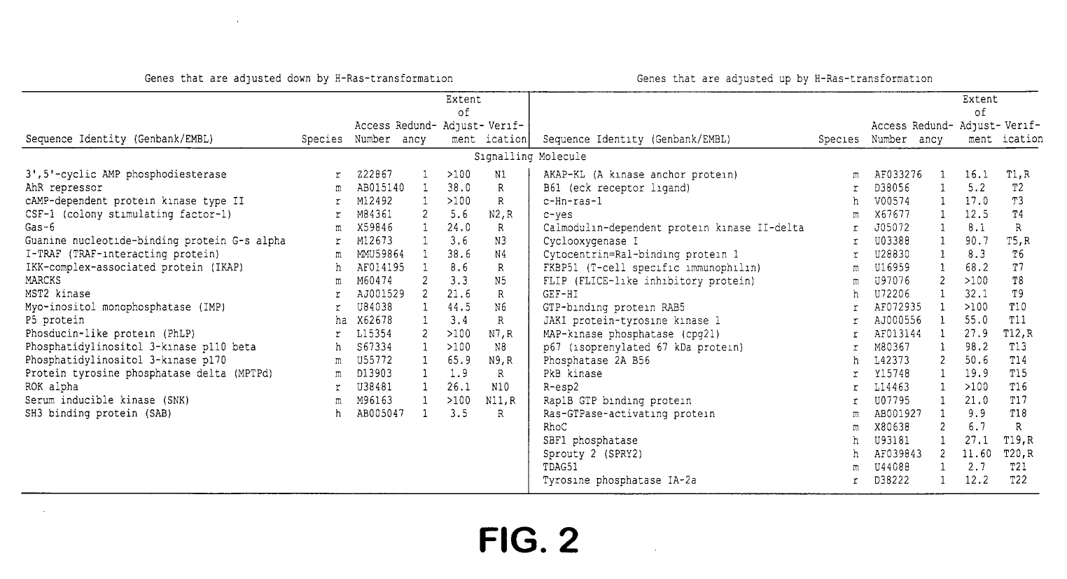

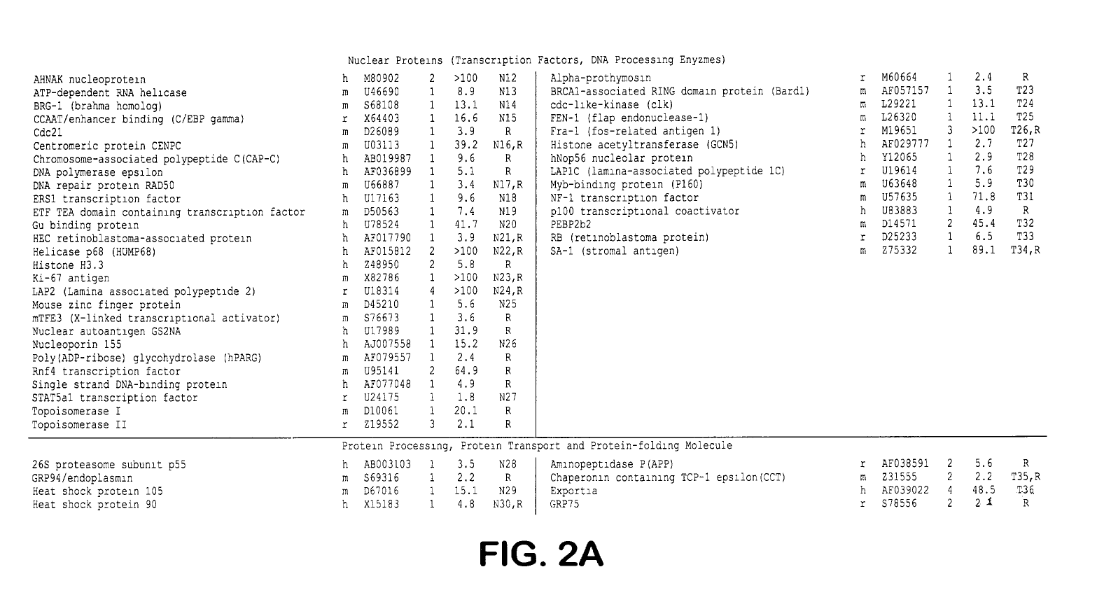

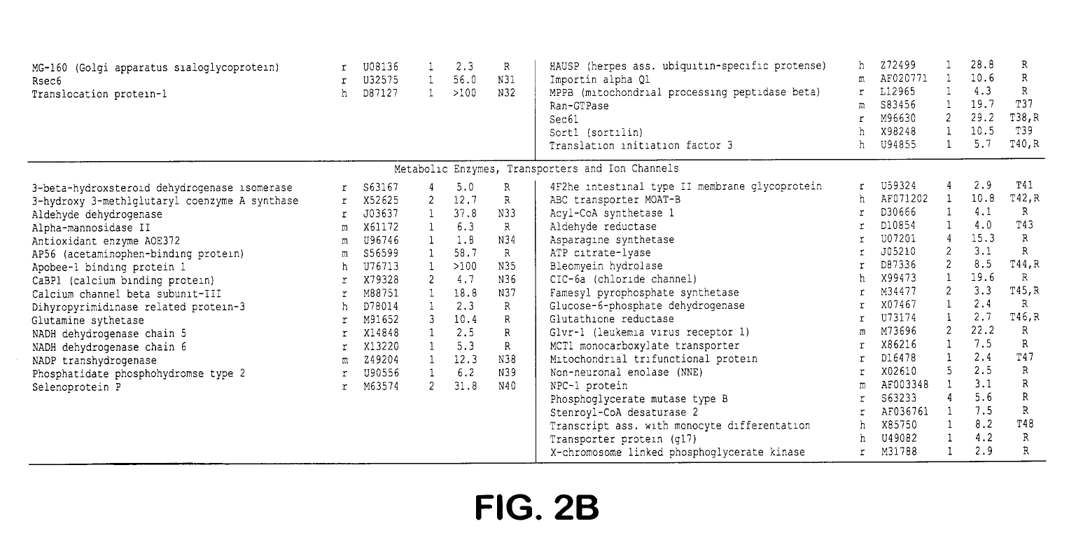

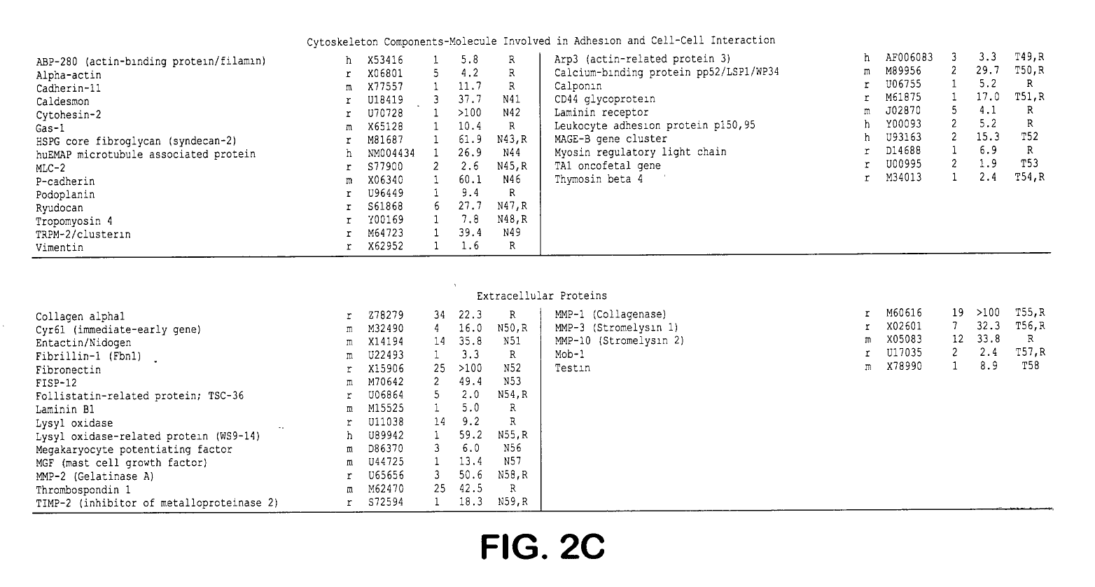

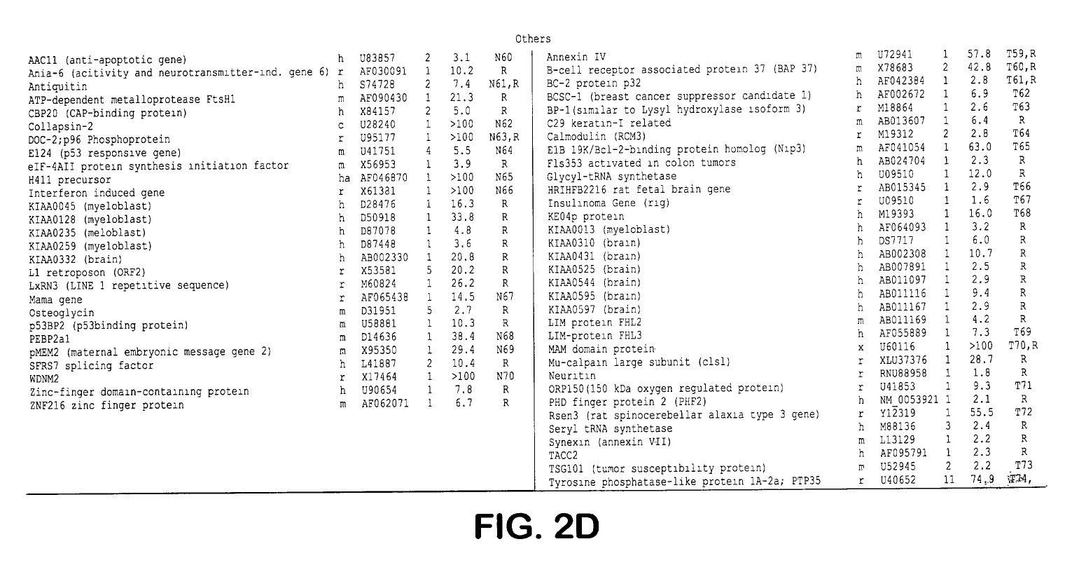

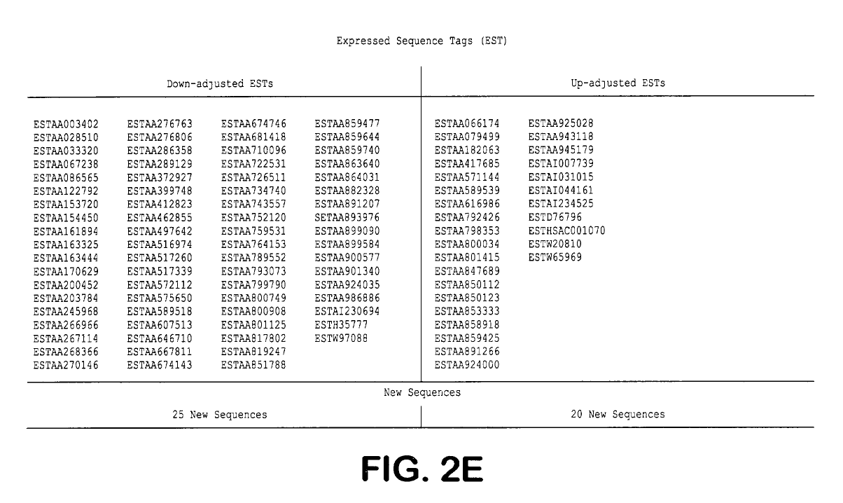

cDNA clones, which preferentially represent mRNAs that are expressed in normal 208F rat cells or in transformed FE-8 cells, were obtained from two subtracted cDNA libraries. For isolation from sequences (N-clones) that are adjusted down in the transition from normal to transformed state, tester cDNA from normal 208F fibroblasts and driver cDNA from transformed FE-8 cells (forward-subtraction) were used. To obtain sequences (T-clones) up-adjusted during the neoplastic transformation, FE-8 cDNA was used as a tester and 208F cDNA was used as a driver (backward-subtraction). The nucleotide sequences of 1357 subtracted cDNA clones were determined after T/A cloning and bacterial transformation. In this case, 823 individual sequences were identified (FIGS. 1, 11). To verify the differential expression by independent methods, subtracted cDNA sequences were amplified by PCR with use of Nested Adapter Primers. The PCR products were separated by gel-electrophoresis and transferred onto hybridization membranes. In each case, two membranes were hybridized with radiolabeled samples from normal 208F cells or transformed FE-8 cells (Reverse Northern Analysis, FIG. 6). In addition, whole RNA from 208F and FE-8 cells with Standard Northern Blots was analyzed with use of individual cDNA fragments as samples. By conventional Northern Analysis, the differential expression of 48 to 50 randomly selected CDNA fragments (96%) on Reverse Northern Blots was verified. In addition, 193 known gene fragments, which yielded no clear signals or even no hybridization signals on the Reverse Northern Membranes, were analyzed in a conventional way. The results of all Standard Northern Blot analyses with reference to differential expression of Ras-transformation targets are shown in FIG. 7. Expressed sequence tags and new sequences were not further analyzed, when the sensitivity of the Reverse Northern Blot was not sufficient to verify clearly a differential expression. The list of all differential genes classified according to selected properties of their products is shown in FIG. 2. The methods used for detection of expression differences between normal and transformed makes it possible to isolate strongly and poorly expressed genes. Based on the compensating step contained in the SSH process, an identification of strongly expressed transcripts (e.g., coding for proteins of the cytoskeleton) and mRNAs with a low copy number (e.g., coding for transcription factors) was made possible. The cDNA fragments identified in this study represent a significant fragment of the differentially expressed genes in the two cell lines.

2.3 Transcriptional Basis for Abnormal Growth, Invasive and Metastatic Properties in Ras-transformed Cells

A number of genes, of which it was already known that they are present in Ras-transformed cells in altered mRNA amounts, was obtained from FE-8 cells. The potential Ras targets, which have a stimulated or de novo expression, comprise the genes coding the metastasis-associated glycoprotein CD44, the transcription factor Fra-1, the alpha-chemokin Mob-1, the metalloproteinases MMP-1 and MMP-3 and the regulatory light chain of myosin. The known down-adjusted Ras targets contain the genes for α-actin, collagen α-1, entactin/nidogen, fibronectin, TGFβ-stimulated sequence TSC36, lysyloxidase, smooth muscle-myosin-light chain (MLC)-2 and NAD-dehydrogenase.

The transcription pattern of Ras-transformed cells showed a single correlation with agressive tumor behavior. Thus, for example, the expression of the laminin receptor, MMP-1 (collagenase), MMP-3 (stromelysin-1), MMP-10 (stromelysin-2) and CD44 glycoprotein, whose importance for metastasizing is known, was stimulated in FE-8 cells. At the same time, a large number of antiproliferative, anti-invasive or antiangiogenic genes was repressed in FE-8 cells. These genes code for syndecan-2, tissue inhibitors of metalloproteases (TIMP)-2, lysyloxidase (rrg-1), thrombospondin-1, protein kinase A II, the myristoylated, alanine-rich C-kinase substrate (MARCKS), and the growth arrest-specific protein GAS-1.

A linkage between the Ras-oncogene-mediated signalling and the pharmaceutical agent resistance was also found based on the upward-adjustment of genes in FE-8 cells, which are involved in transport and processing of cytotoxic pharmaceutical agents, including the multispecific anion transporter MOAT-B, the exopeptidase bleomycin hydrolase and the aldehyde reductase. In addition, various genes were identified that are involved in signal transduction processes for the adjustment of mitogenic activity and the survival of cells as well as of genes that influence the reorganizing of the cytoskeleton, the reaction to stress, oxidative phosphorylation, glycolytic energy production and fatty acid oxidation (FIG. 2).

3.4 Sensitivity of Ras-mediated Transcriptional Changes to the Inhibition of the Raf/Mek-Signal Transduction Path

It is known that a number of signal effector proteins interact with the main effector domains of Ras. In addition to the Raf-kinase, the main effector of Ras, Raf-independent mechanisms are also involved in the Ras-mediated transformation (in the overview, see Khosravi et al., Adv. Cancer. Res. 72 (1998), 57-107). It was now examined to what extent the Raf-signal transduction cascade downstream from the Ras influences the gene transcription and the transformed phenotype in FE-8 cells.

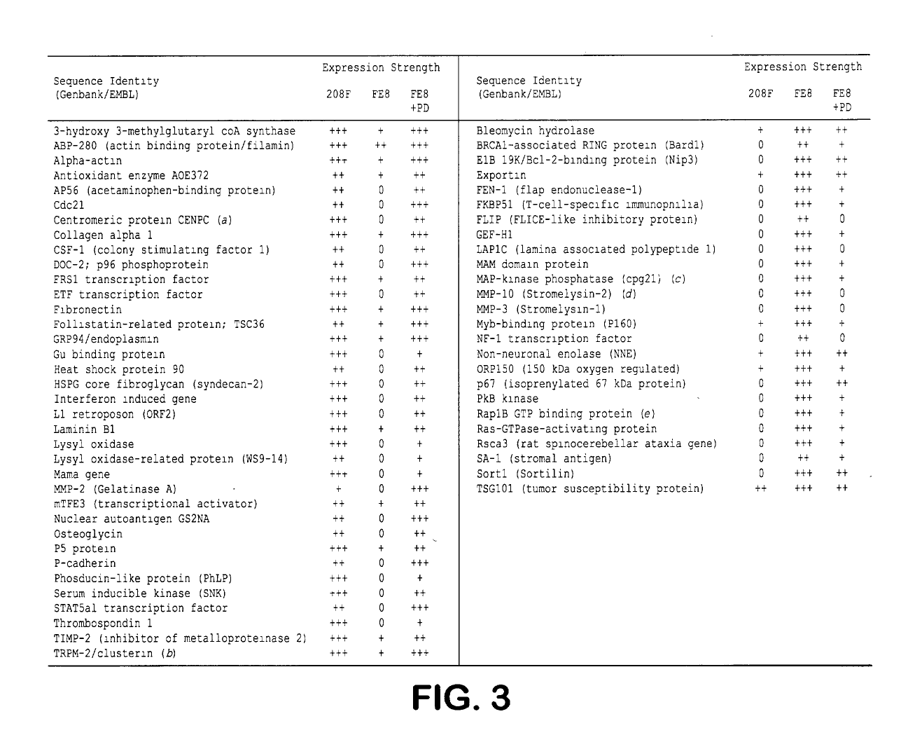

In the treatment with the specific Mek-inhibitor PD98059, FE-8 cells showed a more normal morphology like 208 F-cells (FIG. 5a) and a significantly reduced ability for anchor-independent proliferation (FIG. 5c) despite uniform amounts of p21 Ras (FIG. 5d). The Raf/Mek-signal cascade was blocked, as shown by reduced amounts of phospho-p44/42-MAPK amounts, which could not be distinguished from the non-transformed 208 F-cells (FIG. 5e). cDNA arrays comprising all differentially expressed sequences, which were detectable by Reverse Northern analysis (FIG. 2), were hybridized with radiolabeled samples of RNA from untreated and inhibitor-treated FE-8 cells. In addition, a total of 77 preferentially expressed known genes, which were not detectable either in DNA arrays in a positive manner or in arrays, were studied by conventional Northern Blot analysis (FIGS. 8a to e). In this case, 61 known transcripts were identified, which were sensitive relative to a MAP-kinase inhibition (FIG. 3, FIGS. 8a-e). The H-Ras-mediated-downward-adjustment was reversed for 36 transcription targets, while the upward-adjustment of 25 targets was blocked. The mRNA amounts of 116 genes or expressed sequences was not influenced in FE-8 cells treated with inhibitors.

3.5 Ras Isoform-specific Gene Expression Profiles

The oncogenes H-Ras, K-Ras, and N-Ras and their products resemble one another in structure and function. The Ras proteins differ considerably, however, in the amino acid composition of the C-terminus, the expression pattern and their post-translational modification (for an overview, see Malumbres and Pellicer, Frontiers in Biosciences 3 (1998), 887-912). In addition, individual isoforms are preferably mutated in different types of cancer (Bos, Canc. Res. 49 (1998), 4682-4689).

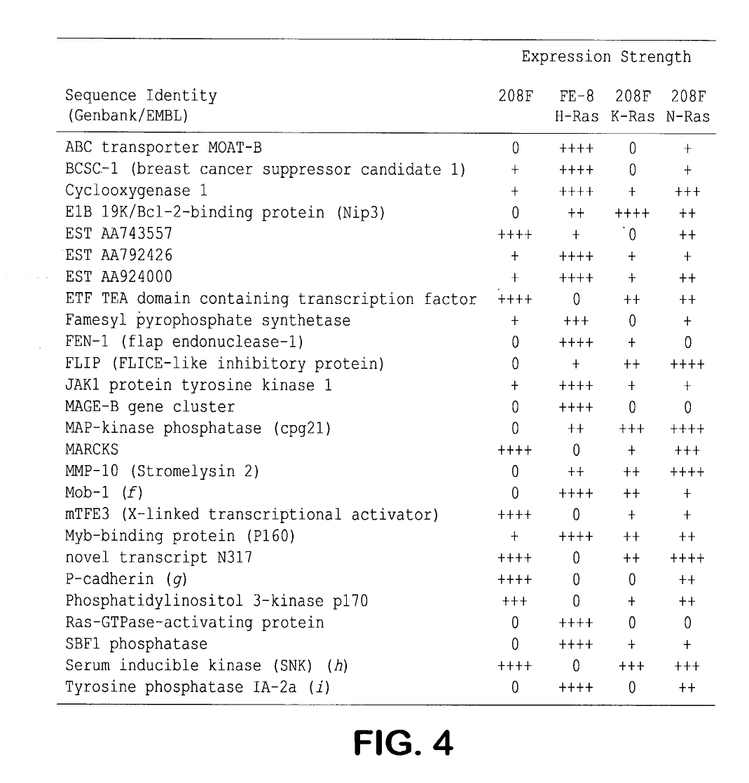

To find out in what way the transcription of the H-Ras target genes influences the two other Ras isoforms, 208F rat cell lines were produced, which express the activated K-Ras or N-Ras gene. These cell lines showed similar properties in the neoplastic transformation such as FE-8 cells (FIG. 9). Radiolabeled cDNA samples of 208F cells transformed by mutated K-Ras or N-Ras were hybridized with the CDNA arrays that contain the H-Ras transformation-sensitive sequences (n=233). The results of a Reverse Northern Analysis were verified by conventional Northern Blot. In addition, 30 poorly expressed genes were analyzed with Northern Blots (FIGS. 8f to i). About 90% of all sequences that are sensitive relative to an H-Ras transformation show a very similar expression pattern in cells, which had been transformed by the two mutated Ras isoforms. The amounts of 26 cDNA fragments showed clear differences, however (FIG. 4, FIGS. 8f-e). More specific H-Ras targets than K-Ras or N-Ras targets were found.

The high degree of similarity of target genes in three independent transfixed cell lines, which express various Ras isoforms, shows that the transcriptional changes can be reproduced to a large extent and are not based on random differences between the cell lines. To study cells of a different tissue type, a randomly selected subclass of target genes was analyzed by s-transformed rat ovarian surface epithelial cells (FIG. 10, left). The transcriptional changes in FE-8 and K-Ras-transformed ROSE cells were very similar. In addition, most of the transcriptional changes in 208F cells that were specific to the FE-8 cells and that had been transformed with an IPTG-inducible H-Ras gene were reproducible (FIG. 10, right).

1. Nucleic acid, characterized in that it shows a differential expression in tumor cells and normal cells, comprising
(a) one of the nucleic acid sequences shown in FIG. 11,
(b) partial sequences thereof with a length of at least 50, preferably at least 100, and especially preferably at least 200 nucleotides,
(c) a sequence that hybridizes with a sequence that consists of (a) and/or (b) under stringent conditions, and/or
(d) a sequence that is complementary to a sequence that consists of (a), (b) and/or (c).

2. Nucleic acid according to claim 1, wherein
the tumor cells are selected from rat fibroblasts that are transformed with H-Ras, N-Ras and K-Ras.

3. Nucleic acid according to claim 1 or 2, wherein
the normal cells are selected from 208F rat fibroblasts.

4. Nucleic acid according to one of claims 1 to 3, wherein
it shows an enhanced expression in a Ras-transformed cell line in comparison to a non-transformed cell line or is expressed de novo in a Ras-transformed cell line.

5. Nucleic acid according to one of claims 1 to 4, wherein
its expression in Ras-transformed cells is adjusted down.

6. Nucleic acid according to claim 4, wherein
it comprises one of sequences N1-N297 according to FIG. 11 or a sequence that thus hybridizes under stringent conditions.

7. Nucleic acid according to claim 6, selected from the sequences that are not yet described as mRNA in rats:
N100, N103, N104, N105, N106, N107, N109, N110, N111, N112, N113, N114, N115, N116, N117, N118, N12, N120, N121, N122, N124, N125, N126, N127, N128, N129, N13, N130, N131, N133, N134, N135, N136, N137, N138, N139, N14, N140, N141, N143, N144, N145, N146, N147, N148, N149, N150, N151, N152, N153, N154, N156, N157, N158, N16, N160, N161, N162, N163, N164, N165, N166, N167, N168, N169, N17, N170, N171, N172, N173, N174, N175, N176, N177, N178, N18, N180, N181, N182, N183, N184, N185, N186, N187, N188, N189, N19, N190, N191, N192, N193, N194, N195, N196, N197, N198, N199, N2, N20, N201, N202, N203, N204, N205, N206, N207, N208, N209, N21, N210, N211, N212, N213, N214, N215, N216, N217, N218, N219, N22, N220, N221, N222, N223, N224, N225, N225, N227, N228, N229, N23, N230, N231, N232, N234, N236, N237, N238, N239, N240, N241, N242, N244, N246, N247, N248, N249, N25, N250, N251, N252, N253, N254, N255, N256, N257, N258, N259, N260, N261, N262, N263, N264, N265, N266, N267, N268, N269, N270, N271, N272, N273, N274, N275, N276, N277, N278, N278, N279, N28, N280, N281, N282, N283, N284, N285, N286, N287, N288, N289, N290, N291, N292, N293, N294, N295, N296, N297, N30, N32, N35, N38, N4, N44, N46, N51, N55, N56, N57, N59, N60, N62, N64, N65, N68, N69, N71, n73, N74, N75, N76, N77, N78, N79, N80, N81, N82, N84, N86, N87, N88, N89, N90, N91, N92, N93, N94, N96, N97, N98, N99

8. Nucleic acid according to claim 6, selected from sequences for which homologous sequences are described in model organisms, such as mice, chickens, Xenopus, C. elegans, Drosophila, but which are not known in humans:
N103, N105, N112, N113, N115, N116, N121, N127, N128, N13, N14, N151, N16, N163, N164, N17, N182, N184, N185, N189, N19, N199, N2, N20, N212, N225, N241, N249, N252, N257, N264, N269, N289, N29, N296, N30, N38, N4, N56, N57; N59, N60, N64, N65, N68, N69, N74, N9

9. Nucleic acid according to claim 5, wherein
it comprises one of sequences T1-T235 according to FIG. 11 or a sequence that thus hybridizes under stringent conditions.

10. Nucleic acid according to claim 9 selected from the sequences that are not yet described as mRNA in rats:
T1, T100, T101, T102, T103, T104, T105, T106, T107, T108, T109, T110, T111, T112, T113, T114, T115, T116, T118, T191, T12, T120, T121, T122, T123, T124, T125, T126, T127, T128, T129, T130, T131, T134, T135, T136, T137, T14, T140, T141,T142, T144, T145, T146, T147, T148, T149, T150, T151, T153, T54, T155, T156, T157, T158, T159, T160, T163, T164, T165, T168, T169, T17, T170, T171, T172, T173, T174, T175, T177, T178, T179, T18, T180, T181, T182, T183, T184, T185, T186, T187, T188, T189, T19, T190, T191, T192, T194, T195, T196, T197, T198, T199, T2, T20, T200, T201, T202, T203, T204, T205, T206, T207, T208, T209, T210, T211, T212, T213, T214, T215, T216, T217,T218, T219, T220, T221, T222, T223, T224, T225, T226, T227, T228, T229, T230, T23, T232, T233, T234, T235, T236, T237, T238, T239, T24, T241, T242, T243, T244, T245, T247, T248, T249, T25, T250, T251, T252, T253, T27, T28, T29, T3, T31, T34, T35, T36, T37, T39, T4, T40, T42, T46, T48, T49, T50, T52, T58, T60, T61, T62, T63, T65, T66, T68, T69, T7, T70, T73, T76, T77, T78, T79, T79, T8, T81, T82, T83, T84, T85, T86, T87, T88, T9, T90, T91, T92, T94, T95, T96, T97, T99

11. Nucleic acid according to claim 9, selected from sequences for which homologous sequences are described in model organisms such as mice, chickens, Xenopus, C. elegans, drosophila, but which are not known in humans:
T1, T 181, T121, T122, T137, T142, T18, T2, T20, T222, T232, T238, T25, T3, T31, T32, T35, T37, T49, T50, T59, T60, T63, T65, T69, T7, T73, T8,

12. Nucleic acid according to one of claims 1 to 11, wherein
it has a homology to human sequences, especially human ESTs or EST clusters.

13. Nucleic acid according to claim 12, selected from
N1, N10, N101, N102, N103, N104, N108, N109, N11, N112, N116, N12, N121, N122, N125, N126, N128, N129, N13, N131, N132, N134, N136, N137, N14, N142, N144, N148, N149, N151, N152, N154, N156, N158, N159, N160, N163, N165, N17, N175, N18, N180, N181, N182, N183, N186, N187, N188, N189, N192, N196, N198, N199, N20, N202, N204, N205, N207, N209, N21, N212, N213, N215, N218, N22, N228, N234, N235, N238, N242, N243, N248, N249, N250, N252, N253, N255, N256, N257, N26, N260, N261, N263, N264, N266, N267, N270, N271, N275, N28, N280, N283, N289, N29, N294, N3, N30, n31, N32, N34, N35, N36, N38, N39, N4, N40, N42, N43, N44, N45, N46, N48, N49, N5, N50, N51, N55, N58, N59, N61, N62, N65, N7, N70, N71, N74, N75, N77, N78, N79, N80, N81, N82, N85, N89, N92, T10, T100, T103, T105, T109, T11, T111, T116, T117, T118, T119, T120, T121, T124, T125, T129, T132, T133, T137, T138, N139, T14, T141, T143, T144, T146, T147, T148, T15, T153, T156, T159, T16, T160, T162, T163, T166, T17, T170, T172, T174, T175, T176, T182, T183, T185, T186, T188, T189, T19, T191, T192, 193, T196, T2, T20, T202, T204, T205, T208, T21, T211, T212, T215, T216, T217, T219, T222, T223, T224, T225, T226, T227, T230, T232, T235, T237, T238, T239, T240, T243, T244, T245, T25, T250, T251, T253, T27, T3, T31, T34, T35, T36, T37, T38, T40, T42, T43, T44, T45, T47, T48, T49, T50, T54, T58, T59, T6, T60, T61, T62, T64, T66, T67, T68, T69, T72, T73, T74, T75, T76, T80, T82, T86, T88, T89, T9, T94, T96, T98,

14. Nucleic acid according to claim 13, wherein
it represents a human gene, a human cDNA or a partial sequence thereof and wherein the corresponding rat-homologous gene shows a differential expression in tumor cells and normal cells.

15. Nucleic acid according to claim 14, wherein
it comprises one of the sequences shown in FIG. 12.

16. Nucleic acid according to one of claims 1 to 15, wherein
it is arranged as an oligonucleotide or as cDNA on an array.

17. Use of a nucleic acid according to one of claims 1 to 16 or a coded polypeptide thereof as a target for diagnostic or therapeutic purposes.

18. Use according to claim 17 for the production of an agent for tumor diagnosis or tumor therapy.

19. Use according to claim 17 or 18, wherein
the expression of nucleic acid is modulated.

20. Use according to claim 19, wherein
the modulation comprises a gene-therapy treatment of the nucleic acid.

21. Use according to claim 19, wherein
the modulation comprises an administration of antisense-RNA or ribozymes.

22. Use according to claim 17 or 18, wherein
the amount and/or localization of the polypeptide that is coded by the nucleic acid is modulated.

23. Use according to claim 22, wherein
the modulation comprises an administration of the polypeptide or an activator thereof.

24. Use according to claim 22, wherein
the modulation comprises an administration of antibodies directed against the polypeptide or inhibitors of the polypeptide.

25. Process for testing the influence of active substances on the gene expression, wherein
an active substance is added to a cell line, RNA is isolated from the cell line, the RNA is labeled, then hybridized on an array, and then the gene expression profile of the cell line is obtained.

26. Process according to claim 25, wherein
the gene expression profile of cell line (a) that is treated with the active substance is compared to the gene expression profile of a cell line that is not treated with the active substance and/or (b) to the gene expression profile of a cell line that is treated with the active substance but is different.
